(19) United States
(12) Patent Application Publication
(10) Pub. No.: US 2023/0390405 A1
(43) Pub. Date: Dec. 7, 2023

(54) SELECTIVE NERVE CELL DEACTIVATION

(71) Applicants: Shlomo TSURIEL, Elkana (IL); Michael ZILBERSHLAG, Givat-Shmuel (IL)

(72) Inventors: Shlomo TSURIEL, Elkana (IL); Michael ZILBERSHLAG, Givat-Shmuel (IL)

(21) Appl. No.: 18/035,526

(22) PCT Filed: Nov. 11, 2021
(86) PCT No.: PCT/IL2021/051341

§ 371 Date: May 5, 2023

Related U.S. Application Data
(60) Provisional application No. 63/112,165, filed on Nov. 11, 2020.

(51) Int. Cl. A61K 47/64; A61K 41/00; A61K 49/00; A61P 29/02

(52) U.S. Cl. CPC A61K 47/64; A61K 41/0052; A61K 41/0057; A61K 49/0056; A61P 29/02

(57) ABSTRACT

Provided herein are conjugates of retrograde tracers and cell-deactivating agents useful in targeting the nerve cells' body (soma) of neurons that are associated with pain, spasm or tonus, as well as methods of controllable selective deactivating of these nerve cells and devices for executing the methods.

### RELATED APPLICATIONS

This application claims the benefit of priority of U.S. Provisional Patent Application No. 63/112,165, filed on Nov. 11, 2020, the contents of which are incorporated herein by reference in their entirety.

### FIELD AND BACKGROUND OF THE INVENTION

The present invention, in some embodiments thereof, relates to selective nerve cell ablation, and more particularly, but not exclusively, to methods, devices, conjugates and pharmaceutical compositions for selective deactivation of neurons.

The need to rapidly conduct impulses over relatively great distances presents the nervous system with considerable challenges. The typical nerve cell shape is elongated with a narrow efferent axonal appendage extending from the neuronal soma (the cell's body or perikaryon) that in humans may be a meter in length and in larger species even longer. The cylindrical axon extension enables the cell to carry action potentials over distance while minimizing the cytoplasmic volume and cell surface area. However, despite the small axon diameter, the axoplasmic volume can be hundreds to thousands of times greater than the cytoplasmic volume within the neuronal cell body due to the axon length. Because the perikaryon is a major source of biosynthesis for components of the axon, there is an essential requirement to distribute these biomolecules that include neurotransmitters, proteins, and organelles along the length of the axon via axonal transport. In addition, severing an axon does not necessarily harm the nerve cell or ends its function—cell deactivation is therefore effected when the cell body (perikaryon) is harmed.

According to updated studies, about 20% of the adult western population suffers from chronic pain and about 40% of these pains, have neuropathic origin. In addition to pain impact on the quality of life, there is also a heavy economic cost and hundreds of millions of people spend huge sums on pain treatment. Most of the treatments currently offered are chronic, that is, in most cases the patient is required to take pills throughout his life (usually those are analgesics that give a systemic effect on the body and are not aimed specifically at treating the injured neurons). Other treatments require a doctor's visit every few weeks or several months. Many of these treatments have unwanted side effects, a large proportion of the drugs used create dependence on users and can even cause addiction. In addition, in many cases, treatment does not provide adequate response to pain.

Hitherto, the approach to treating pain caused by nerve dysfunction is to silence the injured nerve by injecting it directly or attenuating the entire nervous system response by providing systemic therapy. In both cases, the treatment is long, chronic, expensive, unpleasant and not effective enough. The patient is required to take pills chronically, and large proportion of the drugs create dependence.

Peripheral neurons are nerve cells located outside the central nervous system. These are vital neurons for controlling and activating muscles (motor neurons), sensing the body and the environment (sensory neurons), and activating various internal systems such as blood pressure, gland control, digestive system and more (autonomic neurons). Signals in the motor system come from the brain through the spinal cord to the muscles cause them to contract, while signals in the peripheral sensory system transmit different sensations, such as pain, to the dorsal root ganglia (DRG).

In normal condition, nerve cells (neurons) transmit electrical signals (firing) only as needed and enable proper functioning of the body. However, due to illness or injury, any number of neurons may malfunction, and in some cases produce a neural signal without a stimulus, when they are supposed to be silent. In the sensory system, normally functioning neurons fire in response to an external stimulus such as contact or injury, while malfunctioning neurons may fire regardless of pain or non-painful response and cause chronic neuropathic pain.

In an attempt to treat neuropathic pain, the current treatment includes silencing the nerve cell that causes the symptom by severing the neuron near the sensory location hoping to reduce the pain for a limited time, yet soon thereafter the pain returns and may even worsens. An effective treatment for neuropathic pain may be achieved by killing the entire nerve cell, thereby eliminating this cell from the neurologic system; however, the optimal location to target the nerve cell is at the DRG, where it is impossible to identify, isolate and selectively handling that specific neuron.

In an attempt to treat pain, several preliminary studies performed whole DRG ablation or otherwise whole DRG silencing. For example, Nagda, J. V. et al. [Pain Physician, Jul-Aug 2011;14(4):371-6] set out to determine the safety, success rate, and duration of pain relief of repeat pulsed radiofrequency (PRF) and continuous radiofrequency (CRF) lesioning of the DRG/sacral segmental neurons (SN) in patients with chronic lumbosacral radicular pain. They concluded that repeated pulsed and continuous radiofrequency ablation of the lumbar DRG/SN was safe and effective, in many cases, at least for a certain period of time. The main drawback of this approach is the silencing of a whole DRG rather than only the malfunctioning neurons therein.

Currently, the two primary modalities of radiofrequency application to the DRG used in the clinical practice include continuous RF-DRG (CRF), with electrode temperatures in thermo destructive range, and pulsed RF-DRG (PRF). Although the uncontrolled studies reported the clinical efficacy of both CRF and PRF, the controlled clinical data provided results that were variable depending on the pain syndrome being treated and the mode of RF-DRG used. For CRF, limited evidence of short-term efficacy existed in the treatment of cervi-cobrachial pain, the evidence was inconclusive in the treatment of cervicogenic headaches, and limited evidence against its use existed in the treatment of lumbar radicular pain. The complications reported from CRF were limited mainly to sensory disturbances that were infrequent and self-limiting, and no notable complications of PRF were reported. Although proximity to the DRG was sought in all of the studies of RF-DRG, its exact target, the optimal number of treated segments, and the preferred mode, whether CRF or PRF, are not clear. The main advantage of this approach is excluding from treatment the motor neurons that are not located in the DRG. The main disadvantage of CRF and PRF is that in both the treatment harms not only the pain causing neurons but also other sensory neurons that share the same DRG but innervate non-painful areas, such as in the same dermatome (an area of skin innervated by a certain dorsal root ganglion). Loosing sensory of a whole dermatome is a drastic procedure.

Plasmonic photothermal therapy (PPTT), a type of treatment involving the intravenous or intratumoral injection to introduce gold nanoparticles to cancerous cells and the subsequent exposure to heat-generating near-infrared (NIR) light, is a potentially favorable alternative to traditional treatments of localized tumors such as chemotherapy, radiotherapy, and surgery. The current main concern of PPTT, however, is the feasibility of the treatment in clinical settings. Since PPTT's initial use more than 15 years ago, thousands of studies have been published. Ali, M. R. K. at al. [“Gold-Nanoparticle-Assisted Plasmonic Photothermal Therapy Advances Toward Clinical Application”, J. Phys. Chem. C, 2019, 123, 25, 15375-15393] summarize recent scientific progress, including the efficacy, molecular mechanism, toxicity, and pharmacokinetics of PPTT in vitro with cancer cells and in vivo through mouse/rat model testing, animal clinical cases (such as dogs and cats), and human clinical trials. Given the benefits of PPTT, it is believe that it will ultimately become a human clinical treatment that can aid in the ultimate goal of beating cancer.

Photodynamic therapy (PDT) is a form of phototherapy involving light and a photosensitizing chemical substance, used in conjunction with molecular oxygen to elicit cell death (phototoxicity). PDT is popularly used in treating acne, and is used clinically to treat a wide range of medical conditions, including wet age-related macular degeneration, psoriasis, atherosclerosis and has shown some efficacy in anti-viral treatments, including herpes. It also treats malignant cancers including head and neck, lung, bladder and particular skin. The basic mechanism of photodynamic reaction is for example, when a photosensitizer is in its excited state it can interact with molecular triplet oxygen and produce radicals and reactive oxygen species (ROS) that can kill the cell.

Axonal transport (AT), also called axoplasmic transport or axoplasmic flow, is a cellular process responsible for movement of mitochondria, lipids, synaptic vesicles, proteins, and other organelles to and from a neuron's cell body, through the cytoplasm of its axon, called the axoplasm. AT is also responsible for moving molecules destined for degradation from the axon back to the cell body, where they are broken down by lysosomes.

### SUMMARY OF THE INVENTION

The present disclosure provides a method for selective permanent silencing, deactivation or deactivating of a neuron (nerve cell) that experiences and signals adverse signals, referred to herein as symptoms. Deactivation of a nerve cell by directly attacking the soma of the neuron will most likely cause undesired collateral damage to non-targeted neurons that reside closely with the soma of neuron targeted for deactivation. The method provided herein takes advantage of the considerable distance between the cell body (soma) and the axons' termini. While the neuron can be deactivated permanently by irreversibly disrupting biochemical functioning in the soma rather than in the axons, the axon can be used to introduce a locked or inactive cell-deactivating agent into the cell, which after endocytosis will be transported into the soma by innate axonal transport mechanism. Once the cell-deactivating agent is in the soma, it can be activated to effect cell deactivation (i.e., death, silencing, ablation etc.). The method is therefore based on a conjugate between an activatable (and otherwise inactive) cell-deactivating residue and a retrograde tracer residue, and the method is effected in two steps: in the first step the conjugate is administered in the location of the nervous symptom, and in the second step, once the conjugate is transported to the soma, the location of the remote soma is exposed to activating energy that renders the cell-deactivating residue active.

Thus, according to an aspect of some embodiments of the present invention, there is provided a method for selective deactivating of a nerve cell, which is effected by:
a) locally administering a conjugate at a locus characterized by at least one symptom associated with the nerve cell, wherein:
the conjugate comprises a retrograde tracer residue and an activatable cell-deactivating residue;
the conjugate is capable of undergoing endocytosis by an axon of the nerve cell, the endocytosis is effected at the locus;
the retrograde tracer residue effects retrograde axonal transport of the conjugate to a remote soma of the nerve cell;
the activatable cell-deactivating residue is activatable by an activation energy; and
b) delivering the activation energy to the remote soma, thereby activating the activatable cell-deactivating residue and deactivating the nerve cell.

In some embodiments, the method further includes, subsequent to Step a), allowing a time period to lapse before effecting Step b), thereby allowing said conjugate to reach said remote soma.

In some embodiments, the time period is measured empirically and/or estimated based on the distance between said locus and said remote soma.

In some embodiments, the activation energy is in the form of radiation, and the delivering of the activation energy is effected non-invasively or by a minimally invasive procedure.

In some embodiments, the radiation is characterized by a range of wavelengths, and the cell-deactivating residue is activatable by the radiation.

In some embodiments, the radiation is capable of penetrating tissue surrounding remote soma.

In some embodiments, the delivering is effected by minimally invasive fiber-optic needle probe.

In some embodiments, the nerve cell is a sensory nerve cell.

In some embodiments, the nervous symptom associated with the nerve cell is pain.

In some embodiments, the remote soma is in a dorsal root ganglion (DRG).

In some embodiments, the nerve cell is a motor nerve cell and the symptom is spasm and/or tonus.

In some embodiments, the remote soma is in a spinal location.

In some embodiments, the activation energy is selected from the group consisting of infrared or near infrared radiation, laser light, ultrasound energy, and radiofrequency radiation.

In some embodiments, the retrograde tracer residue is a residue of a retrograde tracer selected from the group consisting of horseradish peroxidase (HRP), dextran, isolectin B4, wheat germ agglutinin (WGA), hydroxystilbamidine (a fluorescent dye), cholera toxin subunit B, a and retrograde viral tracers that can be based on Rabies, Pseudorabies virus herpes family viruses Adeno viruses, Adeno associated viruses and others.

In some embodiments, the cell-deactivating residue is a residue of a cell-deactivating agent selected from the group consisting of a nanoparticle, a cytotoxic agent/drug or a combination thereof.

In some embodiments, the nanoparticle is a gold nanoparticle.

In some embodiments, the conjugate further comprises a fluorescent dye residue suitable for detection of the conjugate in the locus.

According to another aspect of some embodiments of the present invention, there is provided a conjugate that includes a residue of a retrograde tracer and a residue of an activatable cell-deactivating agent, wherein:
the conjugate is capable of undergoing endocytosis by an axon of a nerve cell;
the retrograde tracer residue effects retrograde axonal transport of the conjugate to a remote soma of the nerve cell; and
the activatable cell-deactivating residue is activatable by an activation energy.

In some embodiments, the retrograde tracer residue is a residue of a retrograde tracer selected from the group consisting of horseradish peroxidase (HRP), dextran, isolectin, wheat germ agglutinin (WGA), hydroxystilbamidine (a fluorescent dye), cholera toxin subunit B, a retrograde viral tracers that can be based on Rabies, Pseudorabies virus herpes family viruses Adeno viruses, Adeno associated viruses and other viral based agents.

In some embodiments, the retrograde tracer residue is wheat germ agglutinin (WGA).

In some embodiments, the cell-deactivating residue is a residue of a cell-deactivating agent selected from the group consisting of a nanoparticle, a cytotoxic agent/drug or a combination thereof.

In some embodiments, the nanoparticle is a plasmonic photothermal gold nanoparticle.

In some embodiments, the plasmonic photothermal gold nanoparticle is selected from the group consisting of a gold nanorod, a gold nanoshell, a gold nanocage and a twinned gold nanoparticle.

In some embodiments, the plasmonic photothermal gold nanoparticle is characterized by a diameter less than 100 nm.

In some embodiments, the conjugate further includes a fluorescent dye residue suitable for detection of the conjugate in a bodily site.

In some embodiments, the conjugate includes a photosensitizer residue and a retrograde tracer residue.

In some embodiments, the conjugate includes a photosensitizer residue, a retrograde tracer residue, and a fluorescent dye residue.

In some embodiments, the conjugate includes a plasmonic photothermal nanoparticle residue and a retrograde tracer residue.

In some embodiments, the conjugate includes a plasmonic photothermal nanoparticle residue, a retrograde tracer residue, and a fluorescent dye residue.

According to yet another aspect of some embodiments of the present invention, there is provided a device configured to carry out the method presented herein. The device includes a source of the activation energy; and a probe configured for the delivering.

In some embodiments, the activation energy is selected from the group consisting of NIR light, US energy and RF radiation.

In some embodiments, the probe is a needle for minimally invasive delivery of the activation energy.

In some embodiments, device further includes a fluorescent dye detection elements for locating a conjugate having a fluorescent dye residue suitable for detection of the conjugate in a bodily site.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

When applied to an original property, or a desired property, or an afforded property of an object or a composition, the term “substantially maintaining”, as used herein, means that the property has not change by more than 20%, 10% or more than 5% in the processed object or composition.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents a schematic illustration of three neurons, wherein the neuron in the center is a sensory neuron that is targeted for ablation (deactivating), having soma 11 located in dorsal root ganglion (DRG) 12, and innervating locus of sensory symptom (pain) 15 trough axon (neurite) 13 that lead descending sensory signals from terminals 14 in locus 15 to remote soma, and further showing non-targeted sensory neuron 16 that shares DRG 12 with the targeted neuron but innervating non-symptomatic area, and further showing non-targeted non-sensory neuron 17 that also innervate locus 15 but have its soma locate outside DRG 12; and

FIG. 2 presents a schematic illustration of the first step and the second step of the method for selective deactivation of a targeted nerve cell, according to some embodiments of the present invention, wherein the illustration of the first step is showing locus 21, characterized by at least one symptom associated with nerve cell 22, into which conjugate 23, according to some embodiments of the present invention, is injected, and showing conjugate 23a transported along an axon of targeted nerve cell 22 by axonal transport mechanism into its soma that resides in DGR 26, and showing conjugate 23b also transported along an axon of a non-targeted motor neuron having its soma in non-treated spinal cord location 24 (dashed line denotes the spinal cord), and showing non-targeted sensory neuron 25 having a soma in DGR 26 but does not have conjugate 23 transported thereto, and the illustration of the first step is showing conjugate 23a in the soma of nerve cell 22 (denoted by a dashed line to indicate that this neuron is now deactivated) located in DRG 26, being activated by activation energy 27, which is delivered non-invasively by probe 28 onto DRG 26 but not onto non-treated spinal cord location 24 (dashed line denotes the spinal cord) where conjugate 23b entered the soma of the non-targeted motor neuron, and further showing non-targeted sensory neuron 25 having its soma in DGR 26 but does not have conjugate 23.

### DESCRIPTION OF EMBODIMENTS OF THE INVENTION

The present invention, in some embodiments thereof, relates to selective nerve cell ablation, and more particularly, but not exclusively, to methods, devices, conjugates and pharmaceutical compositions for selective deactivation of neurons.

Before explaining at least one embodiment of the invention in detail, it is to be understood that the invention is not necessarily limited in its application to the details set forth in the following description or exemplified by the Examples. The disclosure is meant to encompass other embodiments or of being practiced or carried out in various ways.

As presented hereinabove, current treatments of chronic pain are not satisfactory, and there is a long felt need for a methodology for treating chronic neuropathic pain that is free from the limitations and disadvantages of presently known approaches.

While conceiving the present invention, the present inventors envisioned a method of targeting a specific nerve cells in the DRG, based on axonal transport (AT). The present inventors conceived that AT can be harnessed to deliver a cell-deactivating agent inside a specific nerve cell from the location of the symptom (e.g., pain) to the location of the perikaryon (e.g., the DRG), at which the cell-deactivating agent would be activated, preferably non-invasively, thereby eliminating (ablating) the entire cell. The inventors envisioned an approach that affects only the misfiring neurons while not affecting neurons that may share the same ganglion but are not involved in the causing the symptom.

While reducing the present invention to practice, the present inventors envisioned a conjugate that includes a residue of a retrograde transport agent that transports molecules from the periphery to the DRG, and a residue of a cell-deactivating (cytotoxic) agent that can be activated controllably in a non-invasive or minimal invasive manner. The present inventors also envisioned a methodology wherein the conjugate is administered at the location of the pain (sensory location), and the activation of the cell-deactivating agent is effected at the DRG, thereby targeting only nerve cell bodies that are associated with the pain (injection to the locus of pain) and only sensory and pain neurons, excluding motor nerve that bypath the DRG.

### A Conjugate

Hence, according to some embodiments of the present invention, there is provided a conjugate, which includes at least a residue of a retrograde tracer (a retrograde tracer residue) and a residue of a cell-deactivating (a cell-deactivating residue).

As is well accepted in the art in the molecular context, and as used herein, the term “residue” refers to a portion, and typically a major portion of a molecular entity, such as molecule, or a part of a molecule having or pertaining to a specific function, which has underwent a chemical reaction and is now covalently linked to another molecular entity. A residue may exhibit the specific function of the parent molecule at least to some extent. Hence, in the context of the present invention, a “retrograde tracer residue” of a conjugate molecule is the portion of the conjugate that confers axonal transport of the conjugate from the periphery to the DRG, while the “cell-deactivating residue” will silence the cell's body once activated, preferably at the DRG.

The residue of a cell-deactivating should be selected or adjusted to be delivered through an axon into body of the neuron (e.g., in the DRG) and selected or adjusted for activation at the location of the body of the neuron (e.g., in the DRG) in a controllable in a non-invasive or minimal invasive manner. This special design that allows the conjugate to be transported retrogradely allows the neuron-deactivating method to be selective with respect to the neuron that causes pain, while not affecting perikaryons in the DRG that are not involved with the symptom. This special design also allows the conjugate to be activated controllably in a non-invasive or minimal invasive manner while excluding the motor neurons from ablation.

The conjugate may optionally include a linking moiety that tethers the retrograde tracer residue and the cell-deactivating residue.

As used herein, the term “moiety” describes a group of covalently bonded atoms that form a part of a chemical compound, which typically has certain functionality.

As used herein, the words “link,”, “linked”, “linkage,” “linker”, “bound” or “attached”, are used interchangeably herein and refer to the presence of at least one covalent bond between species or residues, unless specifically noted otherwise.

As used herein, the term “linking moiety” describes a chemical moiety (a group of atoms or a covalent bond) that links two chemical moieties or residues via one or more covalent, salt, hydrogen, aromatic or other types of bonds. A linking moiety may be a covalent bond, or include atoms that form a part of one or both of the chemical moieties it links, and/or include atoms that do not form a part of one or both of the chemical moieties it links. For example, a peptide bond (amide) linking moiety that links two amino acid residues includes at least a nitrogen atom and a hydrogen atom from one amino acid and at least a carboxyl of the other amino acid. In general, the linking moiety can be formed during a chemical reaction, such that by reacting two or more reactive functional groups, the linking moiety is formed as a new chemical entity which can comprise a bond (between two atoms), or one or more bonded atoms. Alternatively, the linking moiety can be an independent chemical moiety comprising two or more reactive functional groups to which the reactive functional groups of other compounds can be attached, either directly or indirectly.

The positions by which the residues are linked together to form the conjugate presented herein, may optionally be selected such that once the linking moiety is broken, any remaining atoms stemming from the linking moiety on each of the residues, if any, do not substantially abrogate or preclude the biological activity (mechanism of biological activity) or functionality of the residues. In some embodiments, cleavage of the linking moiety restores the biological activity of one or each of the residues which formed the conjugate.

In some embodiments of the present invention, the activity of the retrograde tracer residue (axonal transport activity) is maintained while tethered to the cell-deactivating residue, while the cell deactivating activity of the cell-deactivating residue is trigger only by special activation manner of energy radiation direct to the location of the neuron's soma.

In some embodiments of the present invention, the activity of the retrograde tracer residue (axonal transport activity) is maintained while tethered to the cell-deactivating residue, while the cytotoxic activity of the cell-deactivating residue is suppressed in the conjugated (bound) form, and can be restored upon cleavage of the linking moiety. In such embodiments, the conjugate is a delivery vehicle that transports an inactivated cell-deactivating agent, which can be controllably released from at the targeted location at the DRG.

In some embodiments of the present invention the cell-deactivating residue is inactive in terms of cell-deactivation activity until it is activated. Activation of the cell-deactivating residue includes effecting a molecular change in the conjugate or the cell-deactivating residue (e.g., releasing an active cell-deactivating agent form the conjugate or removing a protective group from the cell-deactivating residue) and stimulating the cell-deactivating residue to deactivate the cell using activation energy.

In some embodiments of the present invention the cell-deactivating agent, inactive in the conjugated form, is released in physiological and/or biochemical conditions found in the cell's body. In such embodiments, the linking moiety is referred to as a biocleavable linking moiety. In the context of some embodiments of the present invention, biocleavable linking moieties are selected so as to break and release the cell-deactivating agent from the conjugate under certain conditions, referred to herein as “cleavage conditions”, which are present in nerve cell's body at the DRG.

In some embodiments, biocleavable linking moieties are selected according to their susceptibility to certain enzymes that are likely to be present at the targeted cell body site where cleavage is intended, thereby defining the cleavage conditions.

Non-limiting examples of biocleavable cleavable linking moieties, according to some embodiments of the present invention, include without limitation, amide, carbamate, carbonate, lactone, lactam, carboxylate, ester, cycloalkene, cyclohexene, heteroalicyclic, heteroaryl, triazine, triazole, disulfide, imine, imide, oxime, aldimine, ketimine, hydrazone, semicarbazone, acetal, ketal, aminal, aminoacetal, thioacetal, thioketal, phosphate ester, and the like. Other linking moieties are defined hereinbelow, and further other linking moieties are contemplated within the scope of the term as used herein. Representative examples of biocleavable moieties include, without limitation, amides, carboxylates (esters), carbamates, phosphates, hydrazides, thiohydrazides, disulfides, epoxides, peroxo and methyleneamines. Such moieties are typically subjected to enzymatic cleavages in a biological system, by enzymes such as, for example, hydrolases, amidases, kinases, peptidases, phospholipases, lipases, proteases, esterases, epoxide hydrolases, nitrilases, glycosidases and the like.

It is noted that the definition of the instantly presented conjugate includes a structural definition (a retrograde tracer residue and a cell-deactivating residue), and a functional definition (capable of undergoing endocytosis and axonal transport, and activatable controllably by an external source of energy); in some embodiment the activation energy is deliverable non-invasive or minimally-invasive manners. Hence, it is further noted herein that any prior art molecule that incidentally falls within the definition of the conjugate provided herein is explicitly excluded from the scope of an instant claim for a conjugate that includes a retrograde tracer residue and a cell-deactivating residue. For instance, molecules that are currently used for neuronal tracing and mapping that have a retrograde tracer residue and a fluorescent dye residue, whereas the fluorescent dye residue is implicated with cell death under some conditions.

Specifically excluded are molecules described in the literature, such as Basbaum, A. I. and Menetrey, D., J Comp Neurol, 1987, 261(2), 306-18, doi: 10.1002/cne.902610211; Menétrey, D. and Basbaum, A. I., J Comp Neurol, 1987, 255(3), 439-50. doi: 10.1002/cne.902550310; Basbaum, A. I., J Histochem Cytochem, 1989, 37(12), 1811-5, doi: 10.1177/37.12.2479673; Gao, X. et al., Biomaterials, 2006, 27(18), 3482-90, doi: 10.1016/j.biomaterials.2006.01.038; Zhang, Y. et al., Sci Rep, 2016, 6, 25794, doi: 10.1038/srep25794; Llewellyn-Smith, I. J. et al., J Comp Neurol, 1990, 294(2), 179-91, doi: 10.1002/cne.902940203; and Katiyar, N. et al., Sci Rep, 2021, 11(1), 2566, doi: 10.1038/s41598-021-81995-x, the contents of which are incorporated herein by reference in their entirety for the purpose of explicit exclusion of any part of the contents.

### Tri-Functional Conjugates

In some embodiments of the present invention, the conjugate exhibits a third functionality in the form of a third moiety, being a residue of a fluorescent dye molecule. In such embodiments, the tri-functional conjugate is configured also for mapping of the location of the soma of the targeted neuron.

Neuron tracing and mapping is carried out, as known in the art, by introducing a fluorescent dye residue tethered to a retrograde tracer residue, into an axon of the neuron of interest. The fluorescent dye reaches the soma of the neuron by axonal transport, and detected in the location of the remote soma (e.g., a DRG), thereby tracing and mapping the neuron of interest.

Adding a florescent functionality to the conjugate allows simple detection of the ganglion where the soma is located, and also report the arrival of the conjugate into the soma. Detection is executed by a suitable medical device, as these are known in the art.

Non-limiting examples of fluorescent dye molecules that are useful in the context of some embodiments of the present invention, include Alexa Flour 647 (AF 647), AF 594, AF 750, AF 790 and all other Alexa Flour dyes, rhodamine, CY5, Dylight fluorescent dyes and other red, far red or near infrared florescent.

### Retrograde Tracer

As discussed herein and known in the art, axonal transport can be afforded by neuronal tracers, also referred to as histochemical tracers, which are compounds typically used to reveal the location of cells and track neuronal projections. A neuronal tracer may be retrograde, anterograde, or work in both directions. A retrograde tracer is taken up in the terminal or along the axon or other neuronal processes and transported to the cell body, whereas an anterograde tracer moves away from the cell body of the neuron. In the context of embodiments of the present invention, a retrograde tracer is used to deliver a cell-deactivating agent from a peripheral location where sensory stimuli are expected, or along the axon, or where an axon was severed, into the cell body in the DRG.

The term “retrograde tracer” refers to a molecule that is characterized by the capacity to effect axonal transport from the periphery to the DRG. Non-limiting examples of retrograde tracers are provided in, for example, Xiangmin Xu. et al., Neuron, 2020, 107(6), 1029-1047; Christine, S. et al., Frontiers in Neuroscience, 2019, 13, 897; Kumar, P., Mater Methods 2019; 9:2713; Lanciego, J. L. et al., Brain Structure and Function, 2020, 225, 1193-1224; and Yao, F. et al., PLoS ONE, 13(10), e0205133, the contents of which is incorporated herein by reference.

Exemplary retrograde tracers include horseradish peroxidase (HRP), dextran, isolectin, isolectin B4 (IB4), cholera toxin subunit B, wheat germ agglutinin (WGA), hydroxystilbamidine (a fluorescent dye), viral based tracers such as RABV, and any known axonal retrograde transport agent or tracer.

In the context of the present invention, the term “retrograde tracer residue” or “a residue of a retrograde tracer” interchangeably refer to the part of the conjugate that confers or allows axonal transport of the conjugate from the locus of administration of the conjugate to the perikaryon.

### Cell-Deactivating Agent

In the context of the present invention, treating cells with a cell-deactivating agent cause the cells to undergo a process that leads to permanent deactivation and essentially chronic irreversible silencing of the cell (abolishment of nervous activity of the cell). Permanent neuron deactivation may be achieved by cell death/killing/necrosis, in which the cell loses membrane integrity or some of its essential elements and components, and dies. The cell-deactivating agent can also activate a genetic program of controlled cell death (apoptosis).

The term “deactivation” in the context of the present invention, refers to the state of a neuron once it has been treated with the conjugate and method provided herein. As used herein, the terms “deactivation” and “deactivating” refer to a permanent, chronic and essentially irreversible result of introducing the conjugate provided herein and effecting the instantly provided method in a neuron. In some embodiments, the terms “deactivation” and “deactivating” may be seen, according to some embodiment so of the present invention, as representing killing or ablating a neuron and/or silencing a neuron, as these neural conditions can be identified, characterized and affirmed by known scientific means. Confirming permanent reticence of a neuron, which can be assayed by known scientific methodologies, can be used to affirm the desired result of introducing the conjugate provided herein into a neuron and effecting the method provided herein.

The terms “cell-deactivating agent”, “a residue of a cell-deactivating agent”, and “cell-deactivating residue”, as used in the context of some embodiments of the present invention, refer to agents that cause cell deactivation either directly or indirectly, when activated in the form of the conjugate, or released from the conjugate, or released form of the conjugate and activated.

The terms “cell-deactivating agent”, “residue of a cell-deactivating agent”, and “cell-deactivating residue” therefore refer to the capacity of the conjugate to effect cell silencing controllably upon activation, namely the conjugate per se is nontoxic to the cell until the cell-deactivating residue is controllably activated (e.g., irradiation of photosensitizers or plasmonic photothermal NPs), or controllably released from the conjugate (e.g., a cytotoxic agent that is inactive as long as it is in the conjugate form), or released from the conjugate in physiological condition in the cell and thereafter controllably and activated.

In the context of the herein-provided conjugate, the terms “cell-deactivating agent”, “residue of a cell-deactivating agent”, and “cell-deactivating residue” also refer to the capacity of the conjugate to be transported through an axon to the perikaryon; namely, a cell-deactivating residue, according to some embodiments of the present invention, is suitable for axonal transport in terms of size and biochemical compatibility. For example, in some embodiments, the cell-deactivating residue should not exceed the diameter of the axon, and therefore is selected to exhibit a radius of no more than 50 nm, 100 nm, 150 nm, 200 nm, or 300 nm.

### Drugs as Cell-Deactivating Agents

A residue of a small molecule drug exhibiting cell-deactivating properties, can be tethered to a retrograde tracer residue to form a conjugate, according to some embodiments of the present invention. The drug molecule is selected such that the cell-deactivating properties are rendered mute when it is in the tethered/conjugated form, and are regained once the molecule is released from the conjugate.

Conjugates such as these can be afforded by synthesizing the conjugate with a labile linking moiety that can be broken by exposure to certain types of energy that can be delivered by non-invasive or minimally invasive means. The linking moiety can also be subjected to other cleavage conditions, such as enzymes and other factors that are found only at the soma of the neuron and not in the neurites (axons).

Exemplary cell-deactivating agents that can form a part of the conjugate, according to some embodiments of the invention, include, but are not limited to Amonafide; Camptothecin; Colchicine; Chlorambucil; Cytarabine; Doxorubicin; 3-(9-Acridinylamino)-5-(hydroxymethyl)aniline; Azatoxin; Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Cirolemycin; Cisplatin; Cladribine; Crisnatol Mesylate; Cyclophosphamide; Dacarbazine; Dactinomycin; Daunorubicin Hydrochloride; Decitabine; Dexormaplatin; Dezaguanine; Dezaguanine Mesylate; Diaziquone; Docetaxel; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Additional non-limiting examples of cell-deactivating agents that can form a part of the conjugate, according to some embodiments of the invention, include, but are not limited to daunorubicin, doxorubicin, N-(5,5-diacetoxypentyl)doxorubicin, anthracycline, mitomycin C, mitomycin A, 9-amino aminopertin, antinomycin, N8-acetyl spermidine, 1-(2-chloroethyl)-1,2-dimethanesulfonyl hydrazine, bleomycin, tallysomucin, and derivatives thereof; hydroxy containing cell-deactivating agents such as etoposide, irinotecaan, topotecan, 9-amino camptothecin, paclitaxel, docetaxel, esperamycin, 1,8-dihydroxy-bicyclo[7.3.1]trideca-4-ene-2,6-diyne-13-one, anguidine, morpholino-doxorubicin, vincristine and vinblastine, and derivatives thereof, sulfhydril containing cell-deactivating agents and carboxyl containing cell-deactivating agents. Additional cell-deactivating agents include, without limitation, an alkylating agent such as a nitrogen mustard, an ethylenimine and a methylmelamine, an alkyl sulfonate, a nitrosourea, and a triazene; an antimetabolite such as a folic acid analog, a pyrimidine analog, and a purine analog; a natural product such as a vinca alkaloid, an epipodophyllotoxin, an antibiotic, an enzyme, a taxane, and a biological response modifier; miscellaneous agents such as a platinum coordination complex, an anthracenedione, an anthracycline, a substituted urea, a methyl hydrazine derivative, or an adrenocortical suppressant; or a hormone or an antagonist such as an adrenocorticosteroid, a progestin, an estrogen, an antiestrogen, an androgen, an antiandrogen, a gonadotropin-releasing hormone analog, bleomycin, doxorubicin, paclitaxel, 4-OH cyclophosphamide and cisplatinum.

### Photosensitizers as Cell-Deactivating Agents

Photosensitizers are molecules which absorb light and as a result transfer energy into another nearby molecule. Upon absorbing photons of radiation from incident light, photosensitizers are able to promote a ground state electron into an excited singlet state. This electron in the excited singlet state then flips in its intrinsic spin state to become an excited triplet state electron. Photosensitizers experience varying levels of efficiency for intersystem crossing at different wavelengths of light based on the internal electronic structure of the molecule.

For a molecule to be considered a photosensitizer, it must impart a physicochemical change upon a substrate after absorbing incident light. Upon imparting a chemical change, the photosensitizer returns to its original chemical form. It is important to differentiate photosensitizers from other agents that exhibit photochemical interactions and may induce cell deactivation in reaction to light, including, but not limited to, fluorescent dyes, photoinitiators, photocatalysts, photoacids and photopolymerization. Photoinitiators absorb light to become a reactive species, commonly a radical or an ion, where it then reacts with another chemical species. Photoacids increase in acidity upon absorbing light and thermally reassociate back into their original form upon relaxing. Photopolymerization can occur directly wherein the monomers absorb the incident light and begin polymerizing, or it can occur through a photosensitizer-mediated process where the photosensitizer absorbs the light first before transferring energy into the monomer species.

In the context of embodiments of the present invention, the term “photosensitizer” refers to a molecule or a residue thereof, that utilize light to enact a chemical change in a substrate that release free radicals or reactive oxygen species that lead to cytotoxic conditions and cell deactivation. In the context of embodiments of the present invention, the substrate includes molecular structures inside a cell, and the chemical change deactivates the cell.

Some dyes and fluorescent agents had been conjugated to retrograde tracers manly for mapping neuronal pathways and for labeling cells. Although fluorescent dyes may theoretically be used for bleaching, and therefore may be seen as photosensitizers, there is a fundamental difference between the two groups. A fluorescent dye is chemically stable, dose not bleach and does not produce radicals or reactive oxygen species. A dye that produces radicals or other reactive species will not suitable for imaging in living cells, and in addition will not be sufficiently stable. On the other hand, a photosensitizer is defined by the capacity to produce radicals or other reactive species once irradiated in order to deactivate target cells.

In some embodiments of the present invention, the conjugate includes a residue of a PDT agent (a photosensitizer residue) and a retrograde tracer residue.

Numerous photosensitizers with absorption peaks spanning the 600-800 nm “therapeutic window” have been and continue to be developed. Structural modifications of the photosensitizers can then be made in order to improve tumor deliverability and retention. Chemical alterations can also enhance the yields of light generated reactive oxygen species. In vivo data suggest that vascular and direct tumor cell damage as well as systemic and local immunological reactions are involved in PDT responsiveness.

Non-limiting examples of photosensitizers of the “1^{st} generation” include photofrin, visudyne, levulan/5-aminolevulinic acid, foscan/temoporfin, metvix, laserphyrin, and allumera. Non-limiting examples of photosensitizers of the “2^{nd} generation” include verteporfin, purlytin 5-aminolaevulinic acid, lutex and foscan. Non-limiting examples of photosensitizers of the “3^{rd} generation” include metallation, metallochlorins and metallo-phthalocyanines and others.

In the context of the present invention, conjugates that include a retrograde tracer residue and a fluorescent dye residue that is not a photosensitizer are excluded from the scope of the present invention.

### Nanoparticles as Cell-Deactivating Agents

In the contact of some embodiments of the present invention, nerve cells can also be deactivated effectively by delivering energy to the cell or its immediate surrounding, whereas the cell-deactivating agent serves as the generator, conductor or transformer of that localized energy; for example, nanoparticles having high photo-thermal conversion properties (plasmonic photothermal nanoparticles) can generate intense localized heat in response to radiation, thereby forming a family of highly effective cell-deactivating agents in the context of the present invention. Photothermally induced cell deactivation can take place via apoptosis or necrosis depending on the radiation dosage, type and irradiation time. It also depends on the subcellular location of the nanoparticles (NPs).

Plasmonic photothermal NPs are particles whose electron density can couple with electromagnetic radiation of wavelengths that are far larger than the particle, due to the nature of the dielectric-metal interface between the medium and the particles: unlike in a pure metal where there is a maximum limit on what size wavelength can be effectively coupled based on the material size.

Photo-thermal therapy (PTT) is a non- or minimally-invasive therapy in which photon energy is converted into heat to deactivate cells. Noble metal nanoparticles, e.g., gold or silver, having specific shapes, absorb light strongly and convert photon energy into heat quickly and efficiently, thereby making them superior contrast agents for cell deactivating purposes. Plasmonic photothermal nanoparticles hold a unique photophysical phenomenon, called localized surface plasmon resonance (LSPR), which is the result of the interaction of nanoparticles with light of resonant frequency. As a result of the absorption of resonant light, the free electrons of the metal exhibit a collective coherent oscillation around the nanoparticle surface. This coherent oscillation induced as a result of the absorption of light in resonance with the incident light is called the localized surface plasmon resonance.

The rationale behind use of metal nanoparticles is that plasmonic photothermal nanoparticles have useful non-radiative photo-thermal properties. The absorbed light is converted into heat through a series of photo-physical processes. Firstly, the absorbed light is quickly converted to heat to form a hot metallic lattice by two processes: electron-electron relaxation occurring on femto-seconds and electron-phonon relaxation occurring on the picoseconds. Hot electron temperatures of several thousand degree kelvin are easily reached in the nanoparticles even with laser excitation powers as low as 100 nJ and the lattice temperature on the order of a few tens of degrees can be achieved. The lattice then cools off by phonon-phonon relaxation. It means the heat is dissipated from the particles into the surrounding environment to heat up the species surrounding the nanoparticles. When the nanoparticles are attached to, or enter cells, the heat can change the function of the cells and even destroy them depending on the amount of heat generated by the hot nanoparticles.

Such fast energy conversion and dissipation can be readily used for the heating of the local environment by using light radiation with a frequency strongly overlapping with the nanoparticle surface-plasmon resonance (SPR) absorption band. For sufficient heating, relatively modest continuous laser light is generally used. Depending on the localized SPR wavelength, the laser light is either in the visible region using, e.g., spherical gold nanoparticles, or in the near infrared (NIR) region using, e.g., NIR-absorbing gold-based nanoparticles. For in vivo applications, NIR is favorable as the NIR light penetrates tissue optimally due to minimal absorption by the major absorbents of water and hemoglobin in the tissue. Thus gold or silver nanospheres (NSs), nanorods (NRs) and nanocages (NCs) have been actively investigated for their potentials as cell-deactivating agents.

It is noted that plasmonic photothermal activity of NPs is governed by both size and shape. For example, particles having the same overall size but a different shape (spheres, rods, cages, etc.) exhibit different plasmonic photothermal activity (heat their environment to a different extent when irradiated with the same light).

Selecting NPs that are be suitable for use as a cell-deactivating residues in the conjugate provided herein is essentially a balance between shape/size and the extinction wavelength, since the plasmonic photothermal activity is a function of the absorption and scattering cross-section of the NPs. While it may be possible to deliver particles by AT having a size of about 120 nm, it may be preferred to use particles smaller than 80 nm for more effective AT. On the other hand, the light penetration of near infrared (NIR; 750 nm and higher) is significantly deeper than light having a wavelength of 520 nm. The optimal plasmonic photothermal activity of gold nanospheres of 20 nm in diameter is observed while irradiating with light at about 520 nm wavelength, about 530 nm for 40 nm, and about 550 nm for 80 nm. It is common to specify the size of a particle of an arbitrary shape and volume in terms of an effective radius, or r_{eff}, which represents the radius of a sphere having a volume equal to that of the particle. Thus r_{eff} defines the volume of the nanorod. Gold nanorods of various r_{eff} ranging from 11 nm to 22 nm heat effectively when irradiated at 730 nm to 850 nm, respectively. These data suggests that in the case of gold NPs (AuNPs), it is more preferred to use nanorods than nanospheres. Another example of NPs suitable for the purpose of cell-deactivating residue and tuned to NIR [symbol] max nm=843 include, without limitation, gold nanoshells having core/shell ratio of 40 nm/70.

Information pertaining to the relations between size, shape and plasmonic effect is available to the artisan of the fields, and can be found in studies such as Jain, P. K. et al., “Calculated Absorption and Scattering Properties of Gold Nanoparticles of Different Size, Shape, and Composition: Applications in Biological Imaging and Biomedicine”, The Journal of Physical Chemistry B, 2006, 110(14), 7238-7248], the contents of which are incorporated herein by reference in their entirety.

Nanoparticles can be selected and/or designed and/or otherwise manipulated to serve as a cell-deactivating residue in the context of the conjugate presented herein. In order to serve in the conjugate as a cell-deactivating residue, the NP should be suitably sized and shaped so the conjugate can enter the axon and be delivered to the perikaryon by the retrograde tracer residue. Hence, some plasmonic photothermal NPs, although capable of deactivating cells, are not suitable to serve as a cell deactivating residue due to their oversize, and are therefore excluded from the scope of the term “cell-deactivating residue”.

In order to serve in the conjugate as a cell-deactivating residue, the NP should also be suitably sized and shaped to possess plasmonic photothermal properties in a suitable irradiation wavelength. In the context of the present invention it is not sufficient for a plasmonic photothermal NP to be excitable by any wavelength—it should be actively deactivating cells when irradiated by light of a certain wavelength or range thereof, such as near IR (NIR), thereby allowing the method to be carried out by minimally invasive or non-invasive manner.

A conjugate that includes a retrograde tracer residue and a nanoparticle residue, wherein the NP does not possess plasmonic photothermal properties, or is not suitably sized and shaped for AT, or is not activatable by minimally invasive or non-invasive activation manner (activation not in the NIR region), is excluded from the scope of the present invention.

Information pertaining cell-deactivating agents in the form of plasmonic photothermal NPs can be found, for example, in Huang, X. et al., Alexandria Journal of Medicine, 2011, 47:1, 1-9; Bocaa, S. C. et al., Cancer Letters, 2011, 311(2), 131-140; Md. Abdulla-Al-Mamun et al., Photochem. Photobiol. Sci., 2009, 8, 1125-1129; Van de Broek, B. et al., ACS Nano, 2011, 5, 6, 4319-4328; and Fan, Z. et al., ACS Nano, 2012, 6, 2, 1065-1073, the contents of which is incorporated herein by reference.

Information pertaining to non-invasive activation of gold nanoparticles (AuNPs), iron oxide nanoparticles (IONPs), and nano-graphene oxide (NGO) in enhancement of ultrasound-induced heat generation can be found in, for example, Beik, J. et al., J Therm Biol., 2016, 62(Pt A), 84-89, the contents of which is incorporated herein by reference.

Information pertaining to non-invasive activation by RF energy of internalized nanoparticles (NPs) that exerts overheating (hyperthermia) of the cell, ultimately ending in cell necrosis, can be found in, for example, Corr, S. J. et al., ,J. Vis Exp., 2013, 78, 50480, the contents of which is incorporated herein by reference.

Information pertaining to non-invasive activation of AuNPs by laser irradiation, can be found in, for example, Ebrahim, H. M. et al., Asian Pac J Cancer Prev., 2019, 20(11), 3369-3376, the contents of which is incorporated herein by reference.

Information pertaining to AuNP-assisted PPTT that displayed encouraging therapeutic results and is transitioning from the in vitro/in vivo studies to the clinical stages, can be found in, for example, Ali, M. R. K. at al. [J. Phys. Chem. C, 2019, 123, 25, 15375-15393], the contents of which are incorporated herein by reference in their entirety.

Any other nanoparticles known in the art that can be tethered to a retrograde tracer molecule and be excited non-invasively to generate cell-deactivating heat inside a nerve cell body are contemplated within the scope of the present invention.

Currently, several cell-deactivating agents of the nanoparticle (NPs) family are under study for cancer treatment. Such NPs are contemplated in the context of the present invention as cell-deactivating agents that can be tethered to a retrograde neuronal tracer to afford a conjugate, according to some embodiments of the present invention. For example, silica-gold nanoshells coated with PEG are studies for laser responsive thermal ablation of solid tumors. Spherical nucleic acid (SNA) AuNPs are studies for targeting glioblastoma multiforme or gliosarcoma cells.

Additional information pertaining to cancer treatment using NPs as cell-deactivating agents can be found in the clinical trial portfolios under identifier Nos. NCT00848042, NCT01679470, NCT02680535, NCT03020017, NCT01270139, NCT02755870, NCT01420588, and NCT02782026, the contents of which is incorporated herein by reference.

The unique properties of gold nanoparticles (AuNPs), their rich surface chemistry, and low toxicity as well as easy methods of synthesis have promoted conjugation of the particles with numerous biomolecules for site-specific delivery. Gold nanoparticles have multiple applications including photoablation, diagnostic imaging, radiosensitization, vaccine development, antioxidant, and multifunctional drug-delivery vehicles. These applications require an increasingly complex level of surface decoration in order to achieve efficacy, and limit off-target toxicity.

The skilled artisan would appreciate the chemical and physical approaches commonly utilized in relation to surface decoration and the powerful system used to indicate success of the conjugation, and utilize the techniques known in the art to prepare conjugates comprising metal NPs, as described, for example, in: Asim Ali, Y. et al., Frontiers in Chemistry, 2020, 8(341), 2296-2646; Heinz, H. et al., Surface Science Reports, 2017, 72(1), 1-58; and Jazayeri, M. H. et al., Sensing and Bio-Sensing Research, 2016, 9, 17-22, the contents of which are incorporated herein by reference in their entirety.

### Alternative Cell-Deactivating Agents

In the contact of some embodiments of the present invention, nerve cells can also be deactivated effectively by a substance that undergoes a reaction in order to become toxic. Triggering the reaction is does at the locus of ablation, and can be done by light as describe above, or by exposure to other factors, such as enzymes.

High intensity focused ultrasound (HIFU) can be used as a noninvasive activation technique for ablation. To improve the efficacy of HIFU ablation, researchers developed poly(lactide-co-glycolide) (PLGA) nanoparticles encapsulating perfluoropentane (PFP) and hematoporphyrin monomethyl ether (HMME) as synergistic agents (HMME+PFP/PLGA). Two-step biotin-avidin pre-targeting technique was applied for the HIFU ablation.

### Exemplary Conjugates

Table 1 below presents a list of exemplary retrograde tracers that can be tethered to any one of the photosensitizers or any one of the AuNPs listed therein.

TABLE 1

| Retrograde Tracers | Photosensitizers | Plasmonic AuNPs |
|---|---|---|
| wheat germ agglutinin (WGA) | Second Generation | Nanorods of various r_{eff} |
| horseradish peroxidase (HRP) | 5-Aminolaevulinic acid | ranging from 11 nm to 22 |
| dextran | Verteporfin | nm that heat effectively |
| isolectin B4 (IB4) | Purlytin | when irradiated with NIR |
| hydroxystilbamidine (a | Foscan | radiation having |
| fluorescent dye) | Lutex | wavelength that ranges |
| cholera toxin subunit B | ATMPn | from 730 nm to 850 nm, |
| a rabies viral retrograde tracer | Zinc phthalocyanine | respectively; and gold |
| a pseudorabies viral retrograde | Naphthalocyanines | nanoshells having core/shell ratio of 40 |
| tracer | Functional groups: nitrophenyl, | nm/70 that heat |
| a herpes viral retrograde tracer | aminophenyl, hydroxyphenyl, | effectively when |
| an adeno viral retrograde tracer | pyridiniumyl | irradiated with NIR |
| | Third Generation | radiation having |
| | Metallation | wavelength of about 843. |
| | Expanded metallo-porphyrins | |
| | Metallochlorins/bacteriochlorins | |
| | Metallo-phthalocyanines | |
| | Metallo- | |
| | naphthocyaninesulfobenzo- | |
| | porphyrazines (M-NSBP) | |
| | Metallo-naphthalocyanines | |

### A Method of Selective Deactivating of Nerve Cells

At the heart of the present invention is a separation between the site of the symptoms, where administration of the conjugate takes place, and the site of nerve cells ablation action, where activation of the cell-deactivating residue of the conjugate takes place. This separation allows the isolation of the nerve cells that are targeted for deactivating from other nerve cells in the same location, and the separation between neurons that innervate the locus characterized by the symptom associated with the nerve cell from neurons that are not involved with the symptom but share the same ganglion, hence the same site of ablation. This separation is achieved by axonal transport of the herein-provided conjugate from the locus of the symptom/administration to the site of activation of the cell-deactivating residue, where the nerve cell bodies undergo ablation.

The terms “cell body/bodies”, “soma/somas”, “perikaryon/perikarya”, and “neurocyton” are used herein interchangeably to refer to the bulbous, non-process portion of a neuron, containing the cell nucleus. In the context of the present invention, in order to deactivate a neuron in an attempt to silence sensory or other signals from an axon of the neuron, the cell-deactivating residue should be activated in the soma of the neuron.

Neuronal axons converge into dense mass of nerve cells somas called ganglia, such as the dorsal root ganglia (DRG), which contain the somas of sensory (afferent) neurons, the cranial nerve ganglia that contain the somas of cranial nerve neurons, and autonomic ganglia contain the somas of autonomic nerves. Unlike somas that congregate in ganglia, axons or other neuronal processes (dendrite or neurite) diverge from the somas and spread out to far locations in the organism, innervating vast areas thereof. Axons can be mapped from the periphery to their soma using neuronal tracers, and in the method provided herein, the methodology takes advantage of the ability to expose only axons that signal the adverse symptom (e.g., pain, spasm, tonus) to the conjugate. The methodology also takes advantage of the ability of retrograde tracers to carry a payload in the form of a cell-deactivating agent to the soma. The methodology also takes advantage of the ability to transmit activating energy in a non-invasive manner to the ganglion while expecting to activate the cell-deactivating mechanism only in the somas that had the conjugate therein, namely only neurons that were sending the signals of the adverse symptom. These features allow the selective ablation only of neurons that were exposed to and internalized the conjugate, even if these neurons reside closely with neurons that innervate other parts of the body.

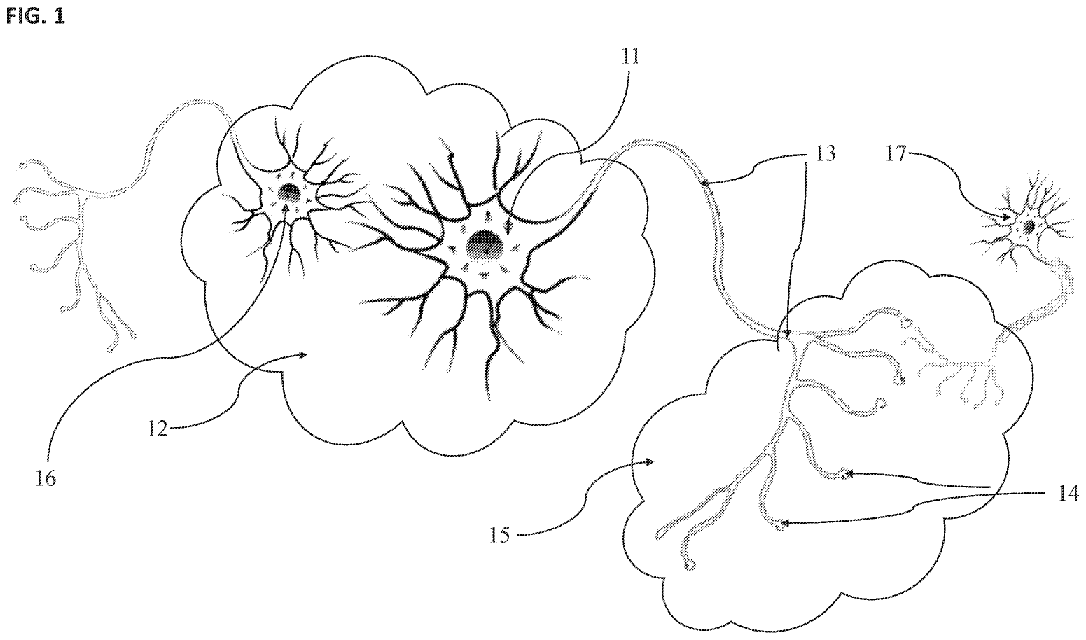

FIG. 1 presents a schematic illustration of three neurons, wherein the neuron in the center is a sensory neuron that is targeted for ablation (deactivating), having soma 11 located in dorsal root ganglion (DRG) 12, and innervating locus of sensory symptom (pain) 15 trough axon (neurite) 13 that lead descending sensory signals from terminals 14 in locus 15 to remote soma, and further showing non-targeted sensory neuron 16 that shares DRG 12 with the targeted neuron but innervating non-symptomatic area, and further showing non-targeted non-sensory neuron 17 that also innervate locus 15 but have its soma locate outside DRG 12.

As can be seen in FIG. 1, sensory neurons that innervate different loci in the body have somas that are located in the same DRG, and motor neurons innervate loci that are innervated by sensory neuron. The method provided herein is designed to selectively deactivate only targeted neurons despite the fact that the targeted neuron have axons where non-targeted motor neurons also have axons, and despite the fact that the remote soma of the targeted neuron shares a ganglion with somas of non-targeted sensory neurons.

Activation of the cell-deactivating mechanism allows targeting only nerve cells that cause the adverse symptoms. The separation into two locally different sites also makes sure that non-targeted nerve cells that innervate the same locus of the symptom, but are not involved with the symptoms, are not affected by the cell-deactivating agent, since their cell bodies are found elsewhere from the cell bodies of the targeted nerve cells. For example, in an embodiment wherein the symptom is neuropathic pain in a limb, the area of the pain is innervated by sensory nerve cells that are targeted, as well as motor nerve cells that are not targeted, however, both types of cells can uptake the conjugate. Since the area of activation of the cell-deactivating mechanism is the DRG, motor cells will not be affected since their cell bodies are found in the spinal cord.

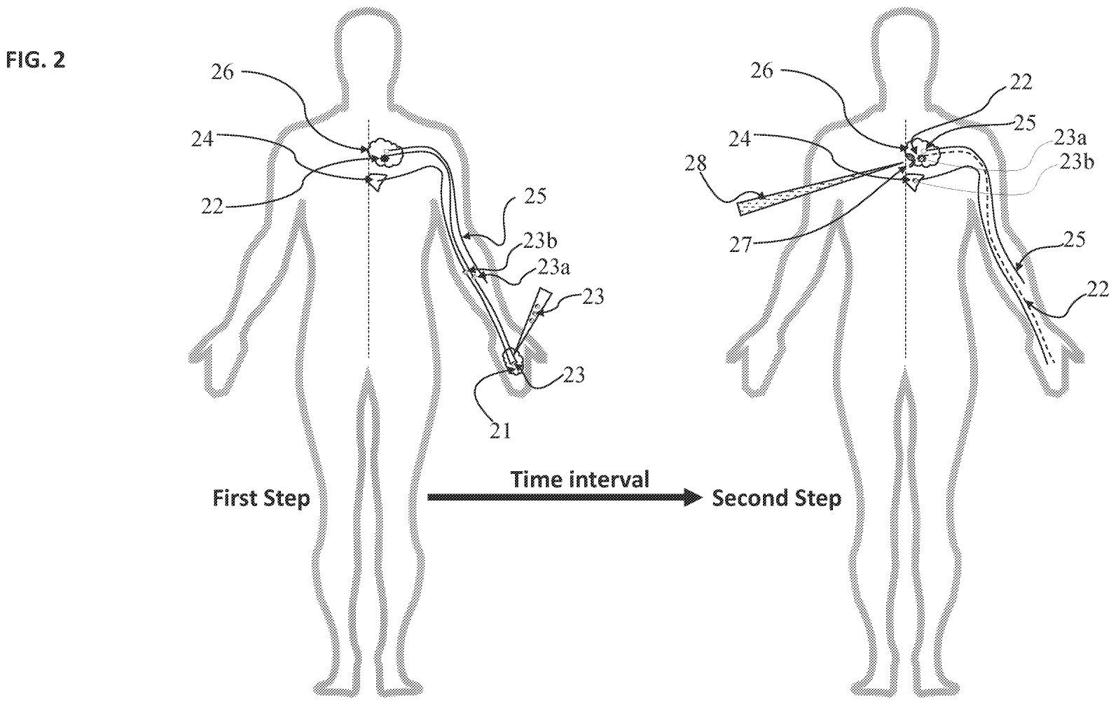

FIG. 2 presents a schematic illustration of the first step and the second step of the method for selective deactivation of a targeted nerve cell, according to some embodiments of the present invention, wherein the illustration of the first step is showing locus 21, characterized by at least one symptom associated with nerve cell 22, into which conjugate 23, according to some embodiments of the present invention, is injected, and showing conjugate 23a transported along an axon of targeted nerve cell 22 by axonal transport mechanism into its soma that resides in DGR 26, and showing conjugate 23b also transported along an axon of a non-targeted motor neuron having its soma in non-treated spinal cord location 24 (dashed line denotes the spinal cord), and showing non-targeted sensory neuron 25 having a soma in DGR 26 but does not have conjugate 23 transported thereto, and the illustration of the first step is showing conjugate 23a in the soma of nerve cell 22 (denoted by a dashed line to indicate that this neuron is now deactivated) located in DRG 26, being activated by activation energy 27, which is delivered non-invasively by probe 28 onto DRG 26 but not onto non-treated spinal cord location 24 (dashed line denotes the spinal cord) where conjugate 23b entered the soma of the non-targeted motor neuron, and further showing non-targeted sensory neuron 25 having its soma in DGR 26 but does not have conjugate 23.

As can be seen in the illustration of the first step in FIG. 2, injecting the conjugate presented herein into a locus characterized by at least one symptom associated with the nerve cell, introduces the conjugate into the axons of both the targeted sensory neuron as well as into axons of non-targeted motor neurons innervating the same locus, but not into axons of non-targeted sensory neurons that do not innervate the locus.

As can further be seen in the illustration of the second step in FIG. 2, activating the conjugate in the DRG where the remote soma of the targeted neuron is located, deactivates the targeted neuron, but does not deactivate other sensory neurons that share the same DRG, and does not deactivate motor neurons that innervate the locus characterized by at least one symptom associated with the targeted neuron. The final result is one targeted and now deactivated (dead) neuron (22), and two unaffected types of neurons, the sensory neuron (25) that does not innervate the symptomatic locus, and the non-sensory neuron that innervates the symptomatic locus but its soma is located outside the treated DRG (26).

Methods of treatments of medical conditions associated with malfunctioning nerve cells stem from the uniqueness of the herein-provided conjugates of retrograde tracers with cell-deactivating agents, such as nanoparticles or photo sensitizers. The conjugate is capable of uptake into the axon followed by migration into the cell body by innate axonal transport, and a suitable mean of activation of the deactivating mechanism is effected at the DRG, or other particular locations where the relevant cell bodies are found, thereby allowing selectively ablation these particular cells.

The intended result of effecting the method provided herein is the elimination or significant reduction of adverse symptoms arising from the targeted neuron, which can be achieved by permanently silencing the neuron, by killing the neuron, or generally deactivating the neuron irreversibly.

Thus, according to an aspect of some embodiments of the present invention, there is provided a method for selective deactivating of a nerve cell; the method is carried out by:
a) locally administering the conjugate, as provided herein, at a locus characterized by at least one symptom associated with the nerve cell, wherein:
the conjugate comprises a retrograde tracer residue and an activatable cell-deactivating residue;
the conjugate is capable of undergoing endocytosis by an axon of the nerve cell, whereas the endocytosis is effected at the locus;
the retrograde tracer residue effects retrograde axonal transport of the conjugate to a remote soma of the nerve cell;
the activatable cell-deactivating residue is activatable by an activation energy; and
b) delivering the activation energy to the remote soma, thereby activating said activatable cell-deactivating residue and deactivating the nerve cell.

In some embodiments, a pharmaceutical composition that includes the conjugate, according to embodiments of the present invention (e.g., AuNPs tethered to a retrograde tracer such as wheat germ agglutinin or dextran), is injected at the bodily site where pain is sensed. The conjugate is taken-up by the ill-affected nerve cells, and is transported up the axons to the nerve cell's body. Activation means (e.g., NIR irradiation, RF, ultrasound or LASER light) are effected at the entire DRG area where the somas are found in order to deactivate only the nerve cells that internalized and transported the conjugate.

The method provided herein is carried out in the above-described steps, whereas the time period that lapses between the steps allows the conjugate to reach the soma of the targeted neuron. This time period that is allowed to lapse, also referred to herein as time interval, generally relates to the length and type of the axon in which the conjugate is being transported. The length of the time period may range from a few hours to a few days and even a week or more.

Generally, axonal transport is characterized by a rate of 1 micrometer per second; hence for the conjugate to reach the DRG from a locus that is 1 meter away from the DRG (e.g. hand palm in an adult human), the time period between the first step and the second step should be about 12 days. In practice, the time period between the first step and the second step can be determined experimentally by the practitioner or from other studies and cases of similar parameters.

According to some embodiments of the present invention, the method of selectively deactivating a nerve cell is effected by administering the conjugates provided herein at the location of an axon of the nerve cell exhibiting adverse symptoms, such as pain, and activating the cell-deactivating residue at the location of the nerve cell body, preferably by non-invasive or minimally invasive manner. The method is effected in essentially two steps:
Step I: In the first step the conjugate is administered, typically by injection, to a bounded area that is associated with the adverse symptoms of pain or spasm; nerve cells that do not innervate this area will not absorb the conjugate. In the area of administration (e.g., injection), the nerve cells absorb the conjugate but this uptake does not affect the cells at this location and stage. The area of administration is referred to herein as the “locus of administration”, which correlates to the “locus characterized by at least one symptom associated with the nerve cell” or the location of the malfunctioning nerve ends. Axons and nerve ends located in the locus of nervous symptom and administration transport the material to the distant cell body (remote soma) by utilizing axonal transport.
Step II: In the second stage the bodily site harboring the somas of the nerve cells that innervate the area of administration where the sensory axons are located, is exposed to the mean of activation of the cell-deactivating agent that forms a part of the conjugate. In some embodiments, mean of activation is NIR, RF, laser light or US, and only the cells that innervate the injected region will be affected and eventually die. The area where the cell-deactivating agent is activated is referred to herein as the “locus of targeted somas”, “a remote soma” or the “locus of activation”.

Once deactivated, sensory nerve cells in the locus of nervous symptom (locus of administration) will no longer signal pain, and motor nerve cells that innervate the same locus and may uptake the conjugate too, will not be deactivated since their cell bodies (remote somas) are located elsewhere from locus of activation (e.g., in the spinal cord), therefor they will not be exposed to the cell-deactivating activation.

A person skilled in the art and practices of the nervous system would appreciate the knowledge and methods for axonal mapping (dermatome map), and would be able to correlate the locus of nervous symptoms to the corresponding DRG, or locus of activation.

In some embodiments, the method is for treating neuropathic pain, phantom pain, spasm, effecting flaccid paralysis, cerebral palsy, and other medical conditions in which selective silencing of nerve cells is beneficial.

In some embodiments of the present invention, the conjugate is injected into the targeted nerve area, e.g.: in the case of motor neurons the ends are in the muscle; in the case of nerve endings sensory neurons are in the skin or in painful tissue. In other cases, with a nerve incision and neuroma formation, the conjugate can be injected directly into the nerve itself, for example to treat phantom pain.

The retrograde neural tracer part of the conjugate is responsible for axonal uptake by the neurons endings and the active transport of the conjugate to the cell body. Once the conjugate reaches the neuronal soma (nerve cell body; for example, DRG in sensory neurons or spinal cord neurons in motoric neurons), this location is endowed with the cell-deactivating agent. In the embodiments where the cell-deactivating agents are plasmonic photothermal nanoparticles, the area of the cell bodies is irradiated with light, ultrasound, radio frequency, or similar, the irradiated energy is absorbed by the nanoparticles that generate heat (or free radicals), and the heat or free radicals causes cell deactivation, while nearby cells, that innervate different area and therefore contain none of the conjugate therein, are not affected.

According to some embodiments of the present invention, a regimen of energy pulses initiate a biochemical process at the end of which only cells that contain the conjugate are deactivated, while the cells and tissue around the affected cells are not affected. The cells containing the conjugate will die within a few hours and the painful area they innervate will cease to be sensed.

### Modes and Tools for Delivering Activation Energy

According to yet another aspect of some embodiments of the present invention, there is provided a device for executing the herein-provided method, which includes a source of activation energy, a probe for delivering the activation energy in a non-invasive or a minimally invasive manner.

The source of activation energy can be any known element that can generate and transmit activation energy of the type discussed hereinabove; for example, a NIR lamp or a NIR laser.

The source of activation energy can be any known element that can generate and transmit high intensity focused ultrasound (HIFU) energy.

The probe for delivering the activation energy can be a fiber-optic needle that can be placed near, on or under the skin of the patient during the delivery of the activation energy.

In some embodiments the device designed for selective ablation of nerve cells in the DRG, which is effected by activating the conjugate that was transported in the axon to the DRG.

In some embodiments the device designed to generate focused energy that can be directed at the DRG and penetrate the tissue surrounding the DRG in a non-invasive or minimal invasive manner.

In some embodiments the device is equipped with a laser generator connected to fiber optic needle probe having light-transfer capability that allow minimal invasive penetration of the tissue surrounding the ganglion for illumination of the entire ganglion where the conjugate has been transported to by AT.

In some embodiments the needle probe is designed to penetrate the skin and tissue and allow close proximity of the light source to the DRG. The Laser generator is designed to generate light in wavelength that activates the cell-deactivating residue of the conjugate, as well as allowing good tissue penetration and lightening of the entire DRG.

According to some embodiments, the device further includes means for tracing and mapping the neuron targeted for deactivating. In some embodiments the device is equipped with an ultrasound imaging element that assist the practitioner in locating the soma containing the conjugate.

In some embodiments the device is equipped with means for detecting the ganglion location by florescent. In such embodiment the conjugate is a tri-functional conjugate, according to some embodiments of the present invention, that includes a residue of a florescent dye' the detection, tracing and mapping methodology are known in the art (see, Background art in the Examples section that follows below). In such embodiments the light source can be a multichromatic laser that generates light energy not only in the conjugate-activation wavelength but also in light in wavelength suitable for fluorescent dye detection.

In some embodiments the device that generate the energy is RF generator, such as, without limitation, the device described in U.S. Pat. No. 7,510,555, and known as the John Kanzius machine. It is noted that the John Kanzius machine should be reconfigured for use in the method provided herein, and adjusted to deliver the RF energy in a more focused manner. For example, the energy transfer between the two conductors that function as parallel-plate capacitor in the John Kanzius machine can be reconfigured to allow aiming the energy to the targeted location. One option is to use conductor plate only in one side, on the back side of the patient, and on the other side of the patient the port of the capacitor is designed as a cone with tip in the size of the conduct area. This design allows focusing the energy to the targeted area (e.g., DRG) while avoiding projection of the RF energy onto the non-targeted surrounding.

### Co-Administration of Anesthetic and Paralysis agents

According to some embodiments of the method for selectively deactivating targeted nerve cells, an agent producing a local peripheral anesthetic effect is co-administered with the conjugate to the peripheral area of interest.

According to some embodiments of the method for treating neuropathic pain, an anesthetic agent is pre-administered or co-administered locally with the conjugate to the area of interest (locus characterized by at least one symptom associated with the nerve cell). An indication for effective treatment would be when the symptoms are affected by the anesthetic agent, in which case the activation of the cell-deactivating residue/agent takes place. The use of a local anesthesia provides, among other uses and purposes, a mean for mapping the exact injection site associate with the malfunctioning axons, a mean for a preliminary validation of the pain source, a mean for a preliminary validation of the administration (injection) area, and a mean for assessing the effect of the method provided herein.

Non-limiting examples of anesthesia agents include lidocaine.

According to some embodiments of the method for treating spasm, a flaccid paralysis agent is co-administered with the conjugate to the area of interest. An indication for effective treatment would be when spasms are affected by the flaccid paralysis agent, in which case the activation of the cell-deactivating residue/agent takes place. The use of a flaccid paralysis agent provides a preliminary validation of the spasm behavior and the correct administration (injection) area.

Non-limiting examples of temporary paralysis agents include botulinum toxin and curare toxin.

### A Pharmaceutical Composition

In some embodiments, the herein-provided conjugate is administered to the locus of targeted neurons, namely the bodily location of the axon of the nerve cell, as a pharmaceutical composition.

Hence, according to an aspect of some embodiments of the present invention, there is provided a pharmaceutical composition which includes as an active ingredient, the conjugate, as provided and described hereinabove.

In some of any of the respective embodiments of the present invention, the pharmaceutical composition is packaged in a packaging material and identified in print, in or on the packaging material, for use in the treatment of a medical condition or a symptom associated with a malfunctioning nerve cell.

The conjugate, according to some embodiments of the present invention, may be incorporated into any suitable carrier prior to use. More specifically, the dose of the conjugate, mode of administration and use of suitable carrier will depend on the locus of nervous symptom associated with the nerve cell, and the locus of remote targeted soma of the nerve cell.

The conjugate may be administered by any conventional approach known and/or used in the art, as long as the conjugate can undergo endocytosis by the nerve cell of interest. Thus, local rather than systemic administration is the most appropriate, such as local injection at the locus of nervous symptom.

The formulations, both for veterinary and for human medical use, of the conjugate according to the present embodiments, typically include such agents in association with a pharmaceutically acceptable carrier, and optionally other therapeutic ingredient(s). The carrier(s) should be “acceptable” in the sense of being compatible with the other ingredients of the formulations and not deleterious to the recipient thereof. Pharmaceutically acceptable carriers, in this regard, are intended to include any and all solvents, dispersion media, coatings, antibacterial and antifungal agents, isotonic and absorption delaying agents, and the like, compatible with pharmaceutical administration. The use of such media and agents for pharmaceutically active substances is known in the art. Except insofar as any conventional media or agent is incompatible with the active ligand, use thereof in the compositions is contemplated. Supplementary active agents, identified or designed according to the invention and/or known in the art, also can be incorporated into the compositions. The formulations may conveniently be presented in dosage unit form and may be prepared by any of the methods well known in the art of pharmacology/microbiology. In general, some formulations are prepared by bringing the active ligand into association with a liquid carrier or a finely divided solid carrier or both, and then, if necessary, shaping the product into the desired formulation.

Pharmaceutical compositions according to some embodiments of the present invention are formulated to be compatible with its intended route of administration. Solutions or suspensions used for the herein intended application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Pharmaceutical compositions suitable for injectable use, according to some embodiments of the present invention, include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS).

Other than the conjugate and a pharmaceutically acceptable carrier, the pharmaceutical composition may also include other agents that have an effect on nerve cells, such as agents producing peripheral axonopathies.

### Uses of the Conjugate and Pharmaceutical Composition

The conjugates provided herein, according to some embodiments of the present invention, allow controlled deactivating of specific nerve cell, and can be used for several applications, as exemplifies below:
Chronic neuropathic pain—due to injury or illness in the sensory system, patients may suffer from chronic pain due to the few nerve cells having axons in the affected area. Using the conjugates and the methods provided herein allows silencing the misfiring sensory cells selectively, thereby treating the pain. Neuropathic pain in general and in particular, diabetic neuropathic pain (DNP), is a chronic condition effecting 2% of the population. In particular, DNP tend to be dual focus (appear in both legs) and as so it is not fit for SCS implantation (new developments of SCS aim to this unmet need). Also, the legs serve by several DRG's that each serve almost the all leg so full DRG ablation is not an option. The method provided herein, according to some embodiment of the present invention, allows focused ablation of the sensory nerve, which will not reduce functionality in the treated area.
Osteoarthritis of the knee—osteoarthritis, and in particular, osteoarthritis of the knee, causes a chronic pain, affecting 3.6% of the population.
Cancer pain—pain in cancer typically arises from a tumor compressing or infiltrating nearby peripheral body parts where the nerve delivers through a DRG. Tumors cause pain by crushing or infiltrating tissue, triggering infection or inflammation, or releasing chemicals that make normally non-painful stimuli painful. Invasion of bone by cancer is the most common source of cancer pain. It is usually felt as tenderness, with constant background pain and instances of spontaneous or movement-related exacerbation and is frequently described as severe.
Phantom pain—deactivating nerve cells that remain active after amputation of a limb alleviate phantom pains.
Involuntarily spasms, convulsions and cramps—In the motor system, neurological injury caused by trauma or illness can be treated by selective deactivating of a number of neurons that act involuntarily and cause spasms.
A substitute for dorsal root rhizotomy—Abnormal nerve in muscles of cerebral palsy (CP) patients can be treated with the presently provided conjugates and methods in dorsal root rhizotomy.
Autonomic system—Disabling nerve cells in the autonomic system to treat medical conditions in the digestive tract, glands, smooth muscles and more.
Cosmetic treatments—Motor nerve paralysis for non-medical purposes, as a substitute the use of botulinum toxin in cosmetic treatments.

The conjugates provided herein, according to some embodiments of the present invention, can be used in basic research where specific cell deactivating in the peripheral and central nervous system is required.

It is expected that during the life of a patent maturing from this application many relevant conjugates will be developed and the scope of the phrase “conjugates” is intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate some embodiments of the invention in a non-limiting fashion.

### Example 1

### Background Art

Tsuriel, S. et al. [“Multispectral labeling technique to map many neighboring axonal projections in the same tissue”, Nature Methods, 2015, 2(6), 547-52], the contents of which are incorporated herein by reference in their entirety, describe a method of mapping the location of axonal arbors of many individual neurons simultaneously via the spectral properties of retrogradely transported dye-labeled vesicles. The study involved injecting overlapping regions of an axon target area with three or more different colored retrograde tracers. On the basis of the combinations and intensities of the colors in the individual vesicles transported to neuronal somata, the projection sites of each neuron's axon was elucidated. This neuronal positioning system (NPS) enables mapping of many axons in a simple automated way. In this study, NPS combined with spectral (Brainbow) labeling of the input to autonomic ganglion cells showed that the locations of ganglion cell projections to a mouse salivary gland related to the identities of their preganglionic axonal innervation. NPS could also delineate projections of many axons simultaneously in the mouse central nervous system.

### Example 2

### RF Activation of AuNPs-WGA Conjugates

Use of wheat germ agglutinin (WGA) conjugate with gold nanoparticles (AuNPs), activated by radio frequency (RF) is described herein.

Lectins are plant molecules that strongly bind to plants pathogens like bacteria and fungi. Wheat germ agglutinin (WGA) is a lectin from wheat that binds fungi chitin. In humans its binds salicylic acid residue that is found on the cell membrane. WGA was not reported to exhibit any negative effects on healthy persons, and no toxicity was shown in intratracheal administration of liposomes attached to WGA.

A WGA-AuNP conjugate includes AuNPs of 5-200 nm in diameter. The conjugate is injected into the locus that is innervated by the neurons fibers targeted for ablation. The WGA retrograde nerve tracer attaches to the receptor in the nerve cells, undergoes endocytosis and transported based on the endogenous axonal transport system retrogradely into the cell body, which is distant from the nerve endings.

After the molecule reaches the cell body, the cell bodies area is radiated by RF energy that cause the conjugate to generate heat, resulting in the deactivation of only the nerve cells that innervate the painful area. Irradiation of AuNPs with RF causes eddy currents and hysteresis losses to generate heat in the immediate vicinity of the AuNPs.

RF irradiation may be effected following the teaching of Dustin E. Kruse, D. E. et al. [“A Radio-frequency Coupling Network for Heating of Citratecoated Gold Nanoparticles for Cancer Therapy: Design and Analysis”, IEEE Trans Biomed Eng., 2011, 58(7), 2002-2012], the contents of which is incorporated herein by reference.

### RF Ablation is Use in Two Different Treatment Form

Currently practiced full ablation: In full ablation the RF probe energizes the DRG and ablate the entire nerve or DRG. The RF probe is configured to generate heat of 80° C. and totally destroy the neurons or kill the DRG's cell bodies. This treatment causes the entire area served by the DRG to loss sensory stimulus entirely. This method is use rarely for extreme pain handling, and is used as third or even last line pain handling solution only.

The focused painful nerve ablation, according to some embodiments of the present invention, allows the deactivating only the nerve serving the painful spot and keep the other nerve untouched.

Currently practiced low temperature ablation/pulsed RF: A very common use of the RF ablation medical device tool for pain treatment is basically using the same ablation tool in pulses that emits low heat about 42° C. just to heat the DRG and control the pain. The DRG mapping of the human body is known and it allow to direct the RF treatment to the right DRG. The mechanism of this pain reduction treatment is not yet understood, and it is assumed that that because the nociceptive (pain) cell bodies are smaller than other sensory cell bodies, they react to RF more than other sensing nerve and as so by heating to 42° C. mainly affects the nociceptive cells and not the sensing cell. In this context by using the herein-provided conjugate and RF pulses to generate low temperature increases in the cells, this treatment may accelerate these phenomena and may effect significant relaxation of pain even in lower heating.

RF generators as a pain management medical device is allowed and commercially available. Non-limiting examples include NeuroTherm NT1000, NeuroTherm NT2000, Stryker Multi-Generator, Cosman G4 RF Generator and LonicRF Generator, all intended for use as an aid in the management of pain in the nervous system by lesioning nerve tissue.

### Example 3

### Laser Activation of AuNPs-WGA Conjugates

This embodiments uses WGA-AuNP conjugates similar to the conjugate described in Example 2, however, the selected AuNPs are plasmonic, exhibiting photothermal properties when activated/irradiated with infrared (IR) or near infrared (NIR) radiation.

The laser use fiber optic attached to a needle to direct the light. The wavelength preferred for activation is NIR about 810 nm, however, the exact wavelength can be optimized to the NPs size and shape. The small diameter spherical NPs are activated by NIR of 400-600 nm, whereas the wavelength for activating nanorods and nanoshells can varies based on the shape and size the NIR target.

In one optional embodiment spherical AuNPs are activated using a sapphire femtosecond laser based on the two-photon concept that allow penetration in tissue using IR wavelength, yet having effective laser operation at 400-500 nm range.

### Example 4

### US Activation of Conjugates

Zhang, Y. et al. [Scientific Reports, 2019, 9, No. 6982], the contents of which are incorporated herein by reference in their entirety, reported that high intensity focused ultrasound (HIFU) can be used as a noninvasive thermal ablation technique for the treatment of benign and malignant solid masses. To improve the efficacy of HIFU ablation, the researchers developed poly(lactide-co-glycolide) (PLGA) nanoparticles encapsulating perfluoropentane (PFP) and hematoporphyrin monomethyl ether (HMME) as synergistic agents (HMME+PFP/PLGA). Two-step biotin-avidin pre-targeting technique was applied for the HIFU ablation. The researchers further modified the nanoparticles with streptavidin (HMME+PFP/PLGA-SA). HMME+PFP/PLGA-SA were highly dispersed with spherical morphology (477.8±81.8 nm in diameter). In the HIFU ablation experiment in vivo, compared with the other groups, the largest gray-scale changes and coagulation necrosis areas were observed in the pre-targeting (HMME+PFP/PLGA-SA) group, with the lowest energy efficiency factor value. Moreover, the microvessel density and proliferation index declined, while the apoptotic index increased, in the tumor tissue surrounding the coagulation necrosis area in the pre-targeting group. Meanwhile, the survival time of the tumor-bearing nude mice in the pre-targeting group was significantly longer than that in the HIFU treatment group. These results suggest that HMME+PFP/PLGA-SA have high potential to act as synergistic agents in HIFU ablation.

In the context of the present invention, the conjugate comprises HMME+PFP/PLGA NPs as described above, and a retrograde tracer such as WGA. This conjugate is injected into the painful area that is innervated by the neurons fibers targeted to ablate. The nerve tracer attaches to the receptor in the nerve cells, undergoes endocytosis and retrogradely transported to the cell body, which is distant from the nerve endings. After the conjugate reaches the cell body, the cell bodies area is radiated by ultrasound waves that cause the conjugate to react and generate heat or mechanical reaction that as result the cells to die.

More enhanced method suggests using heat encapsulate perfluoropentane become toxic as a response to heat. Conjugates comprising such elements can also be used for pain treatment, according to some embodiments of the present invention.

### Example 5

### Photosensitization

According to some embodiments of the present invention a photosensitizer is conjugated with WGA or an alternative neuronal tracer. In the context of treating neuropathic pain, this conjugate is injected into the bodily site where pain is sensed, namely the conjugate is injected at the locus that is been innervated by the neurons which are responsible to the sensation of pain and therefore targeted for ablation, e.g., at a concentration of 10 mg/ml (high concentration). According to a study conducted by the present inventor [Tsuriel, S. et al., Nature Methods, 2015, 2(6), 547-52] and many other works, probe molecules reached the cell body at a concentration similar to the concentration in the injection area but in small vesicles, namely the general concentration inside the cell is much lower but the concentration inside each bubble is high. The dye in the vesicles is very concentrated, however the general concentration of the dye in the cell body is low. These vesicles lysosomes are very acidic with a pH of 4-5 and have decomposition enzymes. The density of photosensitizer in the lysosomes is very similar to the injection density. The nerve tracer attaches to the receptor in the nerve cells undergoes endocytosis and retrogradely transported into the cell body, which is distant from the nerve endings. The length of the axon between the injection area and the DRG is not expected to effect the density but may affect the delay in appearance of the lysosomes in the cell body.

After the conjugate reaches the cell body, the cell bodies area is be radiated by light as in photodynamic therapy (PDT). The light is direct to the DRG using dedicated needle as known in the art.

The photosensitizer in the cell body react to the light, leading to the release free radicals (ROS). The formation of radicals within the lysosomes damages their membranes and they break down. The acid and enzymes are then released and the cell dies (lysosomal cell deactivation by phototoxicity).

Currently several photosensitizers are under investigation or in clinical use, and include, without limitation, Photofrin, Visudyne, Levulan/5-aminolevulinic acid, Foscan/Temoporfin, Metvix, Laserphyrin, and Allumera.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1-30. (canceled)

31. A method for selective deactivating of a nerve cell, comprising:
a) locally administering a conjugate at a locus characterized by at least one symptom associated with the nerve cell, wherein:
said conjugate comprises a retrograde tracer residue and an inactive activatable cell-deactivating residue;
said conjugate is capable of undergoing endocytosis by an axon of the nerve cell, said endocytosis is effected at said locus;
said retrograde tracer residue effects retrograde axonal transport of said conjugate to a remote soma of the nerve cell;
said inactive activatable cell-deactivating residue is activatable by an activation energy;
b) allowing a time period to lapse, thereby allowing said conjugate to reach said remote soma; and
c) delivering said activation energy to said remote soma, thereby activating said activatable cell-deactivating residue and deactivating the nerve cell.

32. The method of claim 31, wherein said time period is measured empirically and/or estimated based on the distance between said locus and said remote soma.

33. The method of claim 31, wherein activation energy is in the form of radiation, and said delivering of said activation energy is effected non-invasively or by a minimally invasive procedure.

34. The method of claim 33, wherein said radiation is capable of penetrating tissue surrounding remote soma.

35. The method of claim 31, wherein the nerve cell is a sensory nerve cell.

36. The method of claim 35, wherein said nervous symptom associated with the nerve cell is pain.

37. The method of claim 35, wherein said remote soma is in a dorsal root ganglion (DRG).

38. The method of claim 31, wherein the nerve cell is a motor nerve cell and said remote soma is in a spinal location.

39. The method of claim 31, wherein said activation energy is selected from the group consisting of infrared or near infrared radiation, laser light, ultrasound energy, and radiofrequency radiation.

40. The method of claim 31, wherein said retrograde tracer residue is a residue of a retrograde tracer selected from the group consisting of horseradish peroxidase (HRP), dextran, isolectin B4, wheat germ agglutinin (WGA), hydroxystilbamidine (a fluorescent dye), cholera toxin subunit B, a and retrograde viral tracers that can be based on Rabies, Pseudorabies virus herpes family viruses Adeno viruses, Adeno associated viruses and others.

41. The method of claim 40, wherein said retrograde tracer residue is wheat germ agglutinin (WGA).

42. The method of claim 31, wherein said cell-deactivating residue is a residue of a cell-deactivating agent selected from the group consisting of a nanoparticle, a cytotoxic agent/drug or a combination thereof.

43. The method of claim 42, wherein said nanoparticle is a plasmonic photothermal gold nanoparticle.

44. The method of claim 43, wherein said plasmonic photothermal gold nanoparticle is selected from the group consisting of a gold nanorod, a gold nanoshell, a gold nanocage and a twinned gold nanoparticle.

45. The method of claim 31, wherein said cell-deactivating residue is a photosensitizer residue.

46. The method of claim 31, wherein said conjugate further comprises a fluorescent dye residue suitable for detection of said conjugate in said locus.

47. A device configured to carry out the method of claim 31, comprising:
a source of said activation energy; and
a probe configured for said delivering.

48. The device of claim 47, wherein said activation energy is selected from the group consisting of near infrared light, ultrasound energy and radio frequency radiation.

49. The device of claim 47, wherein said probe is a needle for minimally invasive delivery of said activation energy.

50. The device of claim 47, further comprising a fluorescent dye detection elements for locating a conjugate having a fluorescent dye residue suitable for detection of the conjugate in a bodily site.
